(19) United States
(12) Patent Application Publication
(10) Pub. No.: US 2004/0146558 A1
(43) Pub. Date: Jul. 29, 2004

(54) Oral enteric-coated preparation

(75) Inventors: Kenji Hirata, Osaka-shi (JP); Masaki Mori, Takino-cho (JP)

Correspondence Address: BACON & THOMAS, PLLC, 625 SLATERS LANE, FOURTH FLOOR, ALEXANDRIA, VA, 22314

(73) Assignee: KYOWA PHARMACEUTICAL CO., LTD., Osaka-shi (JP)

(21) Appl. No.: 10/352,141

(22) Filed: Jan. 28, 2003

(51) Int. Cl. A61K 9/24

(52) U.S. Cl. 424/471

(57) ABSTRACT

An oral enteric-coated preparation of the present invention comprises a core containing an active ingredient unstable to acid, an intermediate film enveloping the core, and an enteric film further enveloping the intermediate film, and the intermediate film comprises a matrix sparsely soluble in water and water-soluble fine particles dispersed therein. The presence of the intermediate film consisting of the matrix sparsely soluble in water and the water-soluble fine particles between the outer enteric film and the core uniformizes the disintegration time of the intermediate film and thus suppresses a variation in the time to start releasing the active ingredient in the core.

### BACKGROUND OF THE INVENTION

1. Field of The Invention

The present invention relates to a stabilized enteric-coated preparation containing an active ingredient unstable to acid (e.g., benzimidazole proton pump inhibitors and the like). In particular, the invention relates to an enteric-coated preparation that begins to release the benzimidazole proton pump inhibitor into intestinal tracts at a time having a relatively low variation.

2. Description of the Related Art

Benzimidazole proton pump inhibitors that intensively suppress secretion of gastric acid have been used, for example, for treatment of gastric and duodenal ulcers. However, active compounds unstable to acid such as benzimidazole proton pump inhibitors (hereinafter, occasionally referred to as acid labile compounds) decompose in an acidic environment. To prevent degradation of the acid labile compound by gastric acid, oral preparations are often coated with a coating film resistant to gastric acid to give enteric film coated preparations. The enteric film is a membrane designed in such a way to be ionized and dissolved under an alkaline condition in the intestinal tract, and the film itself is made of acidic materials. When the acidic materials are made contact with the core containing the acid labile compounds, the acid labile compound may decompose at the interface.

To solve such a problem, an oral preparation consisting of an internal core having an acid labile compound (such as omeprazole, etc.) and additionally an alkalizer, an intermediate film enveloping the core made of an water-soluble material, or a material that decomposes rapidly in contact with water, and an outer enteric film was disclosed in Japanese Unexamined Patent Publications No. S62-258316 and No. S62-258320.

Alternatively, another oral preparation consisting of a core having an acid labile compound, an intermediate film enveloping the core made of a material sparsely soluble in water wherein fine water-insoluble substances were dispersed, and an outer enteric film was disclosed in Japanese Examined Patent Publication No. H7-68125.

### BRIEF SUMMARY OF INVENTION

Among the oral preparations described above, the preparation disclosed in Japanese Examined Patent Publication No. H7-68125 has an advantage in that the active ingredient contained in the core can dissolve rapidly into the intestinal tract once the intermediate film is disintegrated. But, examination by the present inventors revealed that the oral preparation described in Japanese Examined Patent Publication No. H7-68125 had a relatively large variation in the time when the preparation starts releasing the active ingredient since the time is highly sensitive and dependent on thickness of the intermediate film. Additionally, when made thicker, the intermediate film becomes increasingly harder to disintegrate and thus slower to release the ingredient contained in the core, and alternatively when the film becomes thinner, the film cannot sustain its supposed role (i.e., protection of the acid-labile compounds contained in the internal core from the acidic enteric film). In other words, the preparation has a shortcoming in that allowance of the thickness of the intermediate film is narrow. It is difficult to control strictly the thickness of the coating film in the mass-production processes of these oral preparations, and thus further improvement in the enteric-coated preparation is desired.

An object of the present invention is to provide an enteric-coated preparation that has only a small variation in the time when the preparation starts releasing active ingredients into the intestinal tracts.

As a result of intensive studies, the present inventors have found that the use of an intermediate film including a matrix sparsely soluble in water and water-soluble fine particles dispersed therein stabilize the time of releasing medicines (active ingredients) contained in the core, and thus completed the present invention.

Accordingly, the enteric-coated tablet of the present invention is characterized in that it comprises a core containing an active ingredient unstable to acid, an intermediate film enveloping the core, and an outer enteric film enveloping the intermediate film, and that the intermediate film further includes a matrix sparsely soluble in water and water-soluble fine particles dispersed therein.

### BRIEF DESCRIPTION OF DRAWINGS

FIG. 1 is a drawing illustrating the relationship between the content (weight) of intermediate film in tablets and the disintegration time of the intermediate film of the tablets.

### DETAILED DESCRIPTION OF THE INVENTION

An enteric-coated tablet of the present invention comprises a core containing an active ingredient unstable to acid, an intermediate film enveloping the core, and an outer enteric film enveloping the intermediate film.

Further, the present invention is characterized in that the intermediate film includes a matrix sparsely soluble in water and water-soluble fine particles dispersed therein. When prepared by dispersing water-soluble fine particles in the matrix sparsely soluble in water, the resulting intermediate film gives cracks (tears) quite easily upon contact with water. Once the cracks are made, water penetrates through the cracks, and the core become swollen, making the cracks expand wider and penetration of water easier. As a result, the core becomes disintegrated more rapidly, releasing benzimidazole proton pump inhibitors contained therein into the intestinal tracts. Alternatively when prepared from a water-insoluble material dispersed in a matrix sparsely soluble in water, the resulting intermediate film becomes harder and thus slower to give those cracks, and consequently has a larger variation in the time to start disintegration. In contrast, water-soluble fine particles, exerting a weaker influence on the time to start disintegration than water-insoluble substances, even out the same disintegration time.

The water-soluble fine particles are not particularly limited if they are water-soluble and safe to human, but may be selected, for example, from materials having solubility in water at 20° C. of 0.01 g/ml or more, preferably of 0.03 g/ml or more, and more preferably of 0.1 g/ml or more.

Specific examples of the water-soluble fine particles include fine particles of lactose, mannitol, trehalose, sugar, refined sugar, pregelatinized starch, and solid salts of acid [e.g., carbonates (sodium bicarbonate, sodium carbonate, etc.), phosphates (sodium phosphate, etc.), and laurylsulfates (sodium laurylsulfate, etc.)]. The water-soluble fine particles may be used on their own or in combination of two or more kinds of particles. As the solid salts of acid (especially, the solid salts of weak acid) are capable of alkalizing or neutralizing acid, they are effective for stabilization not only of the time to start disintegration of the intermediate film, but of the acid labile compound contained in the core.

The average diameter of the water-soluble fine particles is, for example, 200 μm or less, preferably 100 μm or less, and more preferably about 50 μm or less.

The time when the intermediate film starts to disintegrate (hereinafter, occasionally referred to as disintegration time) can be adjusted by changing the content of the water-soluble fine particles in the intermediate film. The content of the water-soluble fine particles may vary according to the use and/or kind of the preparation, but is, for example, about 1 mass % or more, preferably 5 mass % or more, more preferably 10 mass % or more, and especially 30 mass % or more (e.g., 50 mass % or more) with respect to the sum of the matrix sparsely soluble in water and the water-soluble fine particles. The upper limit of the content thereof may also be decided adequately and is, for example, about 85 mass % or less, preferably about 80 mass % or less, and more preferably about 75 mass % or less. The upper limit described above may be as low as 30 mass % or less, 20mass % or less, or about 10 mass % or less. Presence of an insufficient amount of water-soluble fine particles in the intermediate film often results in extension of the time to start disintegration of the intermediate film and delay in releasing the active ingredient. On the contrary, the presence of an excessive amount of water-soluble fine particles interferes with the film-forming capacity of the matrix sparsely soluble in water, and makes the coating procedures rather difficult.

Although it is possible to control the disintegration time by properly adjusting the amount of the intermediate film in the preparation, the amount of the intermediate film exerts a smaller influence on the disintegration time than the content of the water-soluble fine particles in the intermediate film. Accordingly, even when the amount of the intermediate film applied onto the surface of the core varies due to changes in operational conditions, such as temperature and moisture, of the intermediate film coating process, its influence on the disintegration time and the variation thereof is rather small.

For that reason, according to the present invention, the disintegration time of the intermediate film can be roughly programmed by the amount of the intermediate film in the oral preparation, and the variation in the disintegration time can be accurately adjusted by the content of the water-soluble fine particles in the intermediate film. The amount of the intermediate film may vary according to the use and/or the kind of the preparation, but is, for example, 0.5 mass part or more (preferably 1 mass part or more, more preferably 1.5 mass parts or more), and 20 mass parts or less (preferably 10 mass parts or less, more preferably 6 mass parts or less) with respect to 100 mass parts of the core.

Materials used for the matrix sparsely soluble in water are not particularly limited so long as they have a solubility in water of less than the solubility of the water-soluble fine particles, safe to human, and capable of forming a coating film, but may be selected from slightly soluble materials, very slightly soluble materials, or practically insoluble materials. For example, the materials used for the matrix sparsely soluble in water may have a solubility in water at 20° C. of 1×10⁻³ g/ml or less, preferably about 1×10⁻⁴ g/ml or less.

Specific examples of the material for the matrix include water-insoluble celluloses (e.g., C₂₋₅ alkylcelluloses such as ethylcellulose, etc., cellulose acetate), vinyl alcohol polymers (polyvinyl acetate, polyvinyl alcohol-maleic anhydride copolymers, etc.). The materials for the matrix may be used singly or in combination. The matrix sparsely soluble in water is preferably ethylcellulose.

The intermediate film may further contain other additives as well as the water-soluble fine particles and matrix sparsely soluble in water.

Typical examples of the acid labile compound contained in the core are benzimidazole proton pump inhibitors. Specific examples of the benzimidazole proton pump inhibitor include rabeprazole, omeprazole, pantoprazole, lansoprazole, and alkali metal salts thereof (sodium salts, potassium salts, etc.).

The core typically includes, as well as the acid labile compound (active ingredient) above, excipients (e.g., starches such as corn starch; saccharides such as lactose, sugar, mannitol, sorbitol; crystalline cellulose, talc, etc.), binders (e.g., hydroxypropylcellulose, polyvinylpyrrolidone, etc.), and disintegrators (e.g., lower substituted hydroxypropylcelluloses, microcrystalline cellulose, croscarmellose sodium, crospovidone, etc.). If necessary, the core may additionally contain lubricants (e.g., calcium stearate, magnesium stearate, Carnauba Wax, etc.), surfactants (long chain alkyl sulfonate salts such as sodium laurylsulfate, etc.), and stabilizers (alkalizers, etc.).

The presence of alkalizers in the core is favorable since it prevents degradation of the active ingredient unstable to acid. When the release of the active ingredients and absorption into the body were too slow, disintegrator particles are preferably added to the core. Disintegrator particles with a larger diameter are more effective in preventing the delay in release of the active ingredients. Therefore, though the average diameter of the disintegrator particles may be 55 μm or less, it is preferably 55 μm or more (e.g., 60 μm or more, especially 65 μm or more), and 150 μm or less (e.g., 120 μm or less, especially 100 μm or less). When a plurality of disintegrators are used, either all or a part of the disintegrators may have the average diameters within the favorable range described above.

Examples of the alkalizer include alkali metal salts (especially, salts of weak acids), alkaline earth metal salts (especially, salts of weak acids), alkali metal oxides, alkaline earth metal oxides, etc. Favorable alkali metals are sodium, potassium and the like. Favorable alkaline earth metals are calcium, magnesium, and the like. Examples of the favorable acid (especially, weak acid) include phosphoric acid, carbonic acid, acetic acid, citric acid, aluminometasilicic acid, etc. The alkalizers may be used singly or in combination of two or more.

Favorable alkalizers include carbonates (especially, sodium carbonate, sodium bicarbonate, calcium carbonate), aluminometasilicic acid salts (especially, magnesium aluminometasilicate).

The content of the acid labile compound in the core may vary according to the kind and the use of the acid labile compound, but is preferably, for example, about 1 to 40 mass %, preferably about 5 to 30 mass %, and more preferably 10 to 25 mass %. The acid labile compound usually constitutes the major pharmacologically active ingredient of the preparation of the present invention.

Meanwhile, any of conventional enteric films known in the art may be used as the enteric film of the present invention. Examples of materials used for the enteric film include enteric celluloses (e.g., hydroxypropylmethylcellulose phthalate, hydroxypropylmethylcellulose acetate succinate, carboxymethylethylcellulose, cellulose acetate phthalate, etc.), methacrylic acid-methyl methacrylate copolymers, polyvinyl acetate phthalate, etc. These materials for the enteric film may be used alone or in combination of two or more. Further, the enteric film is not limited to a single layer film and may have a multi-layered structure.

The enteric film preferably contains a plasticizer and/or a processing aid that is used to facilitate coating of the tablet (hereinafter, the processing aid is referred to as coating aid). The plasticizers include, for example, triethyl citrate, Tween 80, triacetin, and the like. The coating aids include, for example, fatty acid glycerol esters, Macrogol, and the like.

The enteric film may also contain colorants, sweeteners, flavors, etc., if desired. Additionally, the enteric-coated preparation may further be sugarcoated.

The shape of the oral preparations of the present invention is not particularly restricted, and any kind of preparations that have been conventionally employed for oral administrations, such as tablet, granule, fine granule, etc., may be used. The preferable preparations include tablets and granules, and preferably tablets. Oral preparations of the present invention can be prepared according to the methods known in the art, by coating a core prepared according to any of the methods known in the art. For preparation of the intermediate film, a coating solution is prepared in advance by dissolving a raw material for the matrix sparsely soluble in water, in an suitable solvent (e.g., ethanol and the like). The coating solution is then applied according to the methods known in the art onto the surface of the core, yielding the intermediate film.

### EXAMPLES

Hereinafter, the present invention will be described in more detail with reference to EXAMPLES and COMPARATIVE EXAMPLES, but it should be understood that the description is intended merely to be illustrative by way of example only and that other modifications, embodiments, and equivalents may be apparent to those skilled in the art without departing from the spirit above or below and scope of the present invention.

In the EXAMPLES and COMPARATIVE EXAMPLES below, the following bare tablet (core) and coating solutions were used

[Bare Tablet Core)]

Ingredients shown in TABLE 1 below were treated in a mixer (“Tumbler Mixer 15” manufactured by DALTON CORPORATION), and the mixture was formulated in a tabletting machine (“VIRGO-19” manufactured by KIKUSUI SEISAKUSYO LTD), yielding bare tablets 1 (Core; 120.0 mg/tablet).

TABLE 1

| Ingredient | Weight (mg) |
|---|---|
| Omeprazole | 20.0 |
| Lactose | 70.0 |
| Corn starch | 21.0 |
| Lower substituted hydroxypropylcellulose | 5.0 |
| Hydroxypropylcellulose | 1.0 |
| Talc | 2.0 |
| Magnesium stearate | 1.0 |
| Total | 120.0 |

[Coating Solutions for Preparation of Intermediate Film]

(1) 5 mass parts of ethylcellulose was dissolved in 95 mass parts of ethanol, and 5 mass parts of lactose (average diameter: 29.7 μm) was added to the resulting solution and dispersed therein to give coating solution 1 for an intermediate film.

(2) 5 mass parts of ethylcellulose was dissolved in 95 mass parts of ethanol, and 5 mass parts of magnesium oxide (average diameter: 10.2 μm) was added to the resulting solution and dispersed therein to give coating solution 2 for an intermediate film.

(3) 10 mass parts of hydroxypropylmethylcellulose was dissolved in 90 mass parts of purified water to give coating solution 3 for an intermediate film.

[Coating Solution for Preparation of Enteric Film]

35 mass parts of hydroxypropylmethylcellulose phthalate, 5 mass parts of a fatty acid glycerol ester (Myvacet 9-40T manufactured by Quest International Co. Ltd.) were dissolved and dispersed in 465 mass parts of an ethanol-water mixed solvent (ethanol 80 vol %) to give coating solution 1 for an enteric film.

Both intermediate and enteric films in the following EXAMPLES and COMPARATIVE EXAMPLES were prepared by means of a tablet coating machine (“Highcoater-mini” manufactured by Freund Industrial Co., Ltd.).

### Example 1

Bare tablets 1 (120 mg/tablet) were coated with the intermediate film-coating solution 1 and dried to give intermediate film coated tablets (122 mg/tablet). The intermediate film coated tablets were further coated with enteric film-coating solution 1 and dried to give enteric-coated tablets (130 mg/tablet).

### Examples 2 to 5

A variety of enteric-coated tablets were prepared in a similar manner to EXAMPLE 1, except that the amount of the intermediate film-coating solution 1 was changed.

### Comparative Example 1

Various enteric-coated tablets were prepared according to the method described in EXAMPLE 1, except that the intermediate film-coating solution 1 was replaced with coating solution 2 for intermediate film (enteric-coated tablet 130 mg/tablet).

### Comparative Examples 2 to 6

Various enteric-coated tablets were prepared in a similar manner to COMPARATIVE EXAMPLE 1, except that the amount of the intermediate film-coating solution 2 is changed.

Weights of the tablets prepared in EXAMPLES 1 to 5 and COMPARATIVE EXAMPLES 1 to 6 are summarized in TABLE 2 below.

TABLE 2

| | Bare tablet | Intermediate | |
|---|---|---|---|
| | weight | tablet | Enteric tablet |
| | (mg) | weight (mg) | weight (mg) |
| EXAMPLE 1 | 120.0 | 122.0 | 130.0 |
| EXAMPLE 2 | 120.0 | 120.8 | 128.8 |
| EXAMPLE 3 | 120.0 | 121.5 | 129.5 |
| EXAMPLE 4 | 120.0 | 122.3 | 130.3 |
| EXAMPLE 5 | 120.0 | 124.8 | 132.8 |
| COMPARATIVE EXAMPLE 1 | 120.0 | 122.0 | 130.0 |
| COMPARATIVE EXAMPLE 2 | 120.0 | 121.3 | 129.3 |
| COMPARATIVE EXAMPLE 3 | 120.0 | 121.8 | 129.8 |
| COMPARATIVE EXAMPLE 4 | 120.0 | 122.7 | 130.7 |
| COMPARATIVE EXAMPLE 5 | 120.0 | 124.3 | 132.3 |
| COMPARATIVE EXAMPLE 6 | 120.0 | 125.2 | 133.2 |

### Comparative Example 7

Enteric-coated tablets were prepared in a similar manner to EXAMPLE 1, except that the intermediate film-coating solution 1 was replaced with intermediate film-coating solution 3 (enteric-coated tablet: 130 mg/tablet).

### Evaluation Example 1

Enteric-coated tablets prepared in EXAMPLE 1, COMPARATIVE EXAMPLE 1 and COMPARATIVE EXAMPLE 7 were respectively dissolved and stirred in the FIRST SOLUTION designated in “disintegration test” of The Japanese Pharmacopoeia (i.e., artificial gastric juice, prepared by dissolving sodium chloride 2.0 g in a mixture of hydrochloric acid 7.0 mL and water, and further diluting the resulting solution with water to volume of 1,000 mL), and the change in appearance of the resulting solutions were determined. Detailed experimental conditions were same as those described in the “disintegration test”.

The results were summarized in TABLE 3.

TABLE 3

| | Hour | | |
|---|---|---|---|
| Sample | 2 | 4 | 6 |
| EXAMPLE 1 | − | − | − |
| COMPARATIVE EXAMPLE 1 | − | − | − |
| COMPARATIVE EXAMPLE 7 | − | +− | +− |

−: no change,
+−: slight yellowing

As apparent from TABLE 3, the solution containing the tablet prepared in COMPARATIVE EXAMPLE 7 began to change in visual appearance after 4 hours of stirring, while the solutions containing tablets prepared in EXAMPLE 1 and COMPARATIVE EXAMPLE 1 did not show any change in appearance for 6 hours of stirring.

### Evaluation Example 2

Disintegration times of the enteric-coated tablets prepared in EXAMPLE 1, COMPARATIVE EXAMPLE 1, and COMPARATIVE EXAMPLE 7 were determined by stirring the respective tablets in the SECOND SOLUTION designated in “disintegration test” of The Japanese Pharmacopoeia (i.e., artificial intestinal juice, prepared by dissolving 250 mL of 0.2 mol/L potassium dihydrogen phosphate in a mixture of 118 mL of 0.2 mol/L sodium hydroxide and water, and further diluting the mixture with water to volume of 1,000 mL). Detailed experimental conditions were same as those described in the “disintegration test”. The results are summarized in TABLE 4.

TABLE 4

| | | Variation |
|---|---|---|
| | Disintegration | in disintegration |
| | time (min) | time (SD, min) |
| EXAMPLE 1 | 5.8 | 0.33 |
| COMPARATIVE EXAMPLE 1 | 5.4 | 0.87 |
| COMPARATIVE EXAMPLE 7 | 4.6 | 0.57 |

SD: Standard Deviation

As apparent from TABLE 4, the enteric-coated tablets prepared in EXAMPLE 1 gave disintegration time similar to those prepared from the tablets of COMPARATIVE EXAMPLE 1 and COMPARATIVE EXAMPLE 7. There was no significant difference observed in the (average) disintegration time among these tablets. Further, the enteric-coated tablets prepared in EXAMPLE 1 had a smaller variation (i.e., standard deviation) in the disintegration time.

### Evaluation Example 3

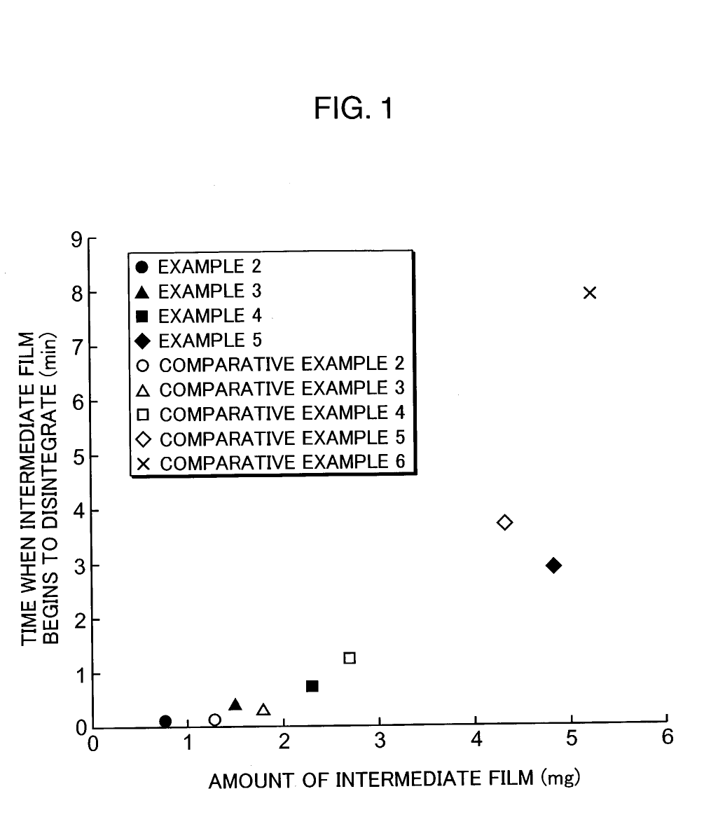

The intermediate coated tablets prepared in EXAMPLES 2 to 5 and COMPARATIVE EXAMPLES 2 to 6 were used here for evaluation. These intermediate coated tablets were immersed and standed still respectively in water at about 25° C., and the time when the intermediate film began to disintegrate was determined by visual observation. The results are summarized in FIG. 1.

As apparent from FIG. 1, the disintegration time of the intermediate coated tablets prepared in COMPARATIVE EXAMPLES increases parabolically as the amount of the intermediate coating film in the oral preparation increases, while the same disintegration time of the tablets prepared in EXAMPLES remains relatively steadfast. That is to say, the tablets prepared in EXAMPLES have a smaller variation in the disintegration time with respect to change in the amount of the intermediate film.

### Preparation Examples 1 to 2

Enteric-coated tablets having compositions shown in TABLES 5 and 6 were prepared in a similar manner to EXAMPLES above.

TABLE 5

| | | Weight |
|---|---|---|
| Classification | Compound | (mg) |
| (Core) | | |
| Acid labile compound | Omeprazole | 20.0 |
| Excipient | Lactose | 72.5 |
| Alkalizers | Sodium bicarbonate | 18.5 |
| Binder | Hydroxypropylcellulose | 2.0 |
| Disintegrator | Crospovidone (average diameter: | 5.0 |
| | 75 μm) | |
| Disintegrator | Lower substituted | 10.0 |
| | hydroxypropylcellulose | |
| Lubricant | Magnesium stearate | 2.0 |
| | Bare tablet total | 130.0 |
| (Intermediate film) | | |
| Water-insoluble matrix | Ethylcellulose | 1.0 |
| Water-soluble fine particles | Sodium laurylsulfate | 1.5 |
| | (average diameter: 11.0 μm) | |
| Water-soluble fine particles | Sodium bicarbonate | 1.5 |
| | (average diameter: 152.4 μm) | |
| | Intermediate coated tablet total | 134.0 |
| (Enteric film) | | |
| Main polymeric ingredient | Hydroxypropylmethylcellulose | 7.9 |
| | phthalate | |
| Coating aid | Fatty acid glycerol ester | 0.8 |
| Colorant | Titanium oxide | 0.3 |
| | Enteric tablet total | 143.0 |

TABLE 6

| | | Weight |
|---|---|---|
| Classification | Compound | (mg) |
| (Core) | | |
| Acid labile compound | Omeprazole | 20.0 |
| Excipient | Lactose | 69.5 |
| Alkalizers | Sodium bicarbonate | 20.0 |
| Binder | Hydroxypropylcellulose | 2.0 |
| Surfactant | Sodium laurylsulfate | 1.5 |
| Disintegrators | Crospovidone (average | 5.0 |
| | diameter: 75 μm) | |
| Disintegrators | Lower substituted | 10.0 |
| | hydroxypropylcellulose | |
| Lubricants | Magnesium stearate | 2.0 |
| | Bare tablet total | 130.0 |
| (Intermediate film) | | |
| Water-insoluble matrix | Ethylcellulose | 1.0 |
| Water-soluble fine particles | Lactose (average | 3.0 |
| | diameter: 29.7 μm) | |
| | Intermediate coated tablet total | 134.0 |
| (Enteric film) | | |
| Main polymeric ingredient | Hydroxypropylmethylcellulose | 7.9 |
| | phthalate | |
| Coating aid | Fatty acid glycerol ester | 0.8 |
| Colorant | Titanium oxide | 0.3 |
| | Enteric tablet total | 143.0 |

Summarizing the present invention described above, the present invention relates to an oral enteric-coated preparation that comprises a core containing an active ingredient unstable to acid, an intermediate film enveloping the core, and an enteric film further enveloping the intermediate film, and that the intermediate film includes a matrix sparsely soluble in water and water-soluble fine particles dispersed therein.

According to the present invention, the presence of the intermediate film including a matrix sparsely soluble in water and water-soluble fine particles between the outer enteric film and the core uniformizes the disintegration time of the intermediate film and thus suppresses the variation in the time to start releasing the active ingredient contained in the core.

As the water-soluble fine particles, compounds such as lactose, mannitol, trehalose, sugar, refined sugar, pregelatinized starch, sodium bicarbonate, sodium carbonate, and sodium phosphate may be used. The content of the water-soluble fine particles is about 1 to 85 mass % with respect to the sum of the matrix sparsely soluble in water and the water-soluble fine particles.

Ethylcellulose is preferable as the matrix sparsely soluble in water.

As the enteric film, hydroxypropylmethylcellulose phthalate, hydroxypropylmethylcellulose acetate succinate, carboxymethylethylcellulose, cellulose acetate phthalate, methacrylic acid-methyl methacrylate copolymers, polyvinyl acetate phthalate or the like may be used. The enteric film more preferably contains a plasticizer and/or a coating aid additionally.

Examples of the acid labile pharmacologically active ingredient contained in the core include benzimidazole proton pump inhibitors such as rabeprazole, omeprazole, pantoprazole, lansoprazole, and alkali metal salts thereof.

The core preferably contains an alkalizer (such as alkali metal salts, alkaline earth metal salts, alkali metal oxides, and alkaline earth metal oxides).

This application is related to Japanese Unexamined Patent Publication No. 2002-234842, the contents of which are hereby incorporated by reference.

What is claimed is:

1. An oral enteric-coated preparation comprising a core containing an active ingredient unstable to acid, an intermediate film enveloping the core, and an outer enteric film enveloping the intermediate film, said intermediate film including a matrix sparsely soluble in water and water-soluble fine particles dispersed therein.

2. An oral enteric-coated preparation according to claim 1, wherein said water-soluble fine particles are particles of at least one material selected from the group consisting of lactose, mannitol, trehalose, sugar, refined sugar, pregelatinized starch, sodium bicarbonate, sodium carbonate, and sodium phosphate.

3. An oral enteric-coated preparation according to claim 1, wherein the content of said water-soluble fine particles is 1 to 85 mass % with respect to the sum of the matrix sparsely soluble in water and the water-soluble fine particles dispersed therein.

4. An oral enteric-coated preparation according to claim 1, wherein said matrix sparsely soluble in water is ethylcellulose.

5. An oral enteric-coated preparation according to claim 1, wherein said enteric film comprises at least one material selected from the group consisting of hydroxypropylmethylcellulose phthalate, hydroxypropylmethylcellulose acetate succinate, carboxymethylethylcellulose, cellulose acetate phthalate, methacrylic acid-methyl methacrylate copolymer and polyvinyl acetate phthalate.

6. An oral enteric-coated preparation according to claim 5, wherein said enteric film further includes a plasticizer and/or a coating aid.

7. An oral enteric-coated preparation according to claim 1, wherein said active ingredient unstable to acid is a benzimidazole proton pump inhibitor.

8. An oral enteric-coated preparation according to claim 7, wherein said benzimidazole proton pump inhibitor is rabeprazole, omeprazole, pantoprazole, lansoprazole, or an alkali metal salt thereof.

9. An oral enteric-coated preparation according to claim 1, wherein said core contains an alkalizer.

10. An oral enteric-coated preparation according to claim 9, wherein said alkalizer comprises at least one alkalizer selected from the group consisting of alkali metal salts, alkaline earth metal salts, alkali metal oxides, and alkaline earth metal oxides.
